(19) United States
(12) Patent Application Publication
(10) Pub. No.: US 2019/0015075 A1
(43) Pub. Date: Jan. 17, 2019

(54) ULTRASONIC DIAGNOSTIC APPARATUS AND CONTROLLING METHOD

(71) Applicant: CANON MEDICAL SYSTEMS CORPORATION, Otawara-shi (JP)

(72) Inventors: Hiroki Yoshiara, Kawasaki (JP); Yu Igarashi, Kawasaki (JP)

(73) Assignee: CANON MEDICAL SYSTEMS CORPORATION, Otawara-shi (JP)

(21) Appl. No.: 16/034,983

(22) Filed: Jul. 13, 2018

(30) Foreign Application Priority Data
Jul. 14, 2017 (JP) 2017-137899

(51) Int. Cl. A61B 8/08; A61B 8/00; A61B 8/06

(52) U.S. Cl. CPC A61B 8/481; A61B 8/54; A61B 8/469; A61B 8/463; A61B 8/06; A61B 8/488; A61B 8/5215

(57) ABSTRACT

According to one embodiment, an ultrasonic diagnostic apparatus includes a transmission/reception circuit and processing circuitry. The transmission/reception circuit is configured to repeatedly perform ultrasonic scanning of an object to which a contrast agent is administered. The processing circuitry is configured to analyze dynamics of the contrast agent in a region of interest in the object based on image data acquired by the ultrasonic scanning. The transmission/reception circuit performs the ultrasonic scanning at a frame rate or a volume rate having a value according to an analysis result of the processing circuitry.

### CROSS-REFERENCE TO RELATED APPLICATIONS

This application claims the benefit of priority of Japanese Patent Application No. 2017-137899, filed Jul. 14, 2017, the entire contents of which are incorporated herein by reference.

### FIELD

Embodiments described herein relate generally to an ultrasonic diagnostic apparatus and a controlling method.

### BACKGROUND

In recent years, ultrasound diagnostic apparatuses have been increasingly performing contrast echo method using an ultrasound contrast agent of an intravenous administration type. This method aims to evaluate blood flow dynamics by, for example, injecting the ultrasound contrast agent from a vein in examination of the heart, liver, or the like, to enhance the blood flow signal. Many of the contrast agents are those in which microbubbles function as reflection sources.

Due to the nature of the delicate substrate of bubbles, the bubbles are destroyed even with ultrasonic irradiation at a normal diagnostic level by the mechanical effect of the irradiation, resulting in a decrease in the signal intensity from the scan surface. Therefore, in order to observe the dynamics of the reflux stream in real time, it is necessary to relatively reduce collapse of bubbles by scanning, e.g., by imaging by ultrasonic transmission at low sound pressure. Since the signal/noise ratio (S/N ratio) is also lowered in such imaging by ultrasonic transmission at low sound pressure, various signal processing methods to compensate for it have been considered. With these imaging methods, it has come to be imaged at a high signal/noise ratio in real time with ultrasonic transmission at low sound pressure. Contrast enhanced ultrasound is used for the examination of micro structures and micro vessel structures which cannot be seen in CT (Computed Tomography) or MRI (Magnetic Resonance Imaging) because of its real time nature and high spatial resolution.

In order to precisely observe the fast inflow process of the arterial predominant phase occurring immediately after administration of the contrast agent, a high temporal resolution imaging method is desired, and a high frame rate imaging method has been developed. Meanwhile, after the inflow of the contrast agent is completed, a normal frame rate is suitable for preventing unnecessary destruction of the contrast agent and for observing the static post vascular phase. Therefore, it is preferable to acquire data at a frame rate having a value according to the dynamics of the contrast agent, i.e., according to whether the contrast agent is in the fast inflow process of arterial predominant phase or in the completion state of the inflow such as post vascular phase. Hence, a technique for switching the frame rate or the volume rate of the ultrasonic scanning at a predetermined time has been proposed.

However, the inflow rate of the contrast agent varies according to the object and the lesion. Therefore, it is difficult by the technique of switching the frame rate at the predetermined time to perform the ultrasonic scanning at a suitable frame rate according to the dynamics of the contrast agent (whether the contrast agent is in the fast inflow process of arterial predominant phase or in the completion state of the inflow such as post vascular phase).

### BRIEF DESCRIPTION OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a block diagram showing an example of a configuration of an ultrasonic diagnostic apparatus according to an embodiment of the present invention;

FIG. 2 is a schematic block diagram illustrating functions of processor of the processing circuitry;

FIG. 3 is a flowchart showing an example of a schematic procedure for changing the frame rate or the volume rate of the ultrasonic scanning according to the dynamics of the contrast agent by the processing circuitry shown in FIG. 1;

FIG. 4 is an explanatory diagram showing an example of a relationship between a time intensity curve and ultrasonic images from immediately after the injection of the contrast agent until a predetermined time elapses;

FIG. 5 is a flowchart showing an example of a procedure implemented by the processing circuitry shown in FIG. 1 for changing the frame rate or the volume rate of the ultrasonic scanning according to the dynamics of the contrast agent immediately after the injection of the contrast agent;

FIG. 6 is an explanatory diagram showing an example of a relationship between an ultrasonic image at each of t=t1, t2, and t3 shown in FIG. 4 and a luminance histogram of a region of interest;

FIG. 7 is an explanatory diagram showing an example of a relationship between TIC and a threshold value Ith of the signal intensity;

FIG. 8 is a diagram for explaining the third inflow completion determination method;

FIG. 9 is a flowchart showing an example of a procedure by processing circuitry shown in FIG. 1 for changing the frame rate or the volume rate of the ultrasonic scanning according to the dynamics of the contrast agent before and after the execution of the flash; and

FIG. 10 is an explanatory diagram showing an example of TIC before and after the execution of the flash.

### DETAILED DESCRIPTION

Hereinbelow, a description will be given of an ultrasonic diagnostic apparatus and a controlling method according to embodiments of the present invention with reference to the drawings.

In general, according to one embodiment, the ultrasonic diagnostic apparatus includes a transmission/reception circuit and processing circuitry. The transmission/reception circuit is configured to repeatedly perform ultrasonic scanning of an object to which a contrast agent is administered. The processing circuitry is configured to analyze dynamics of the contrast agent in a region of interest in the object based on image data acquired by the ultrasonic scanning. The transmission/reception circuit performs the ultrasonic scanning at a frame rate or a volume rate having a value according to an analysis result of the processing circuitry.

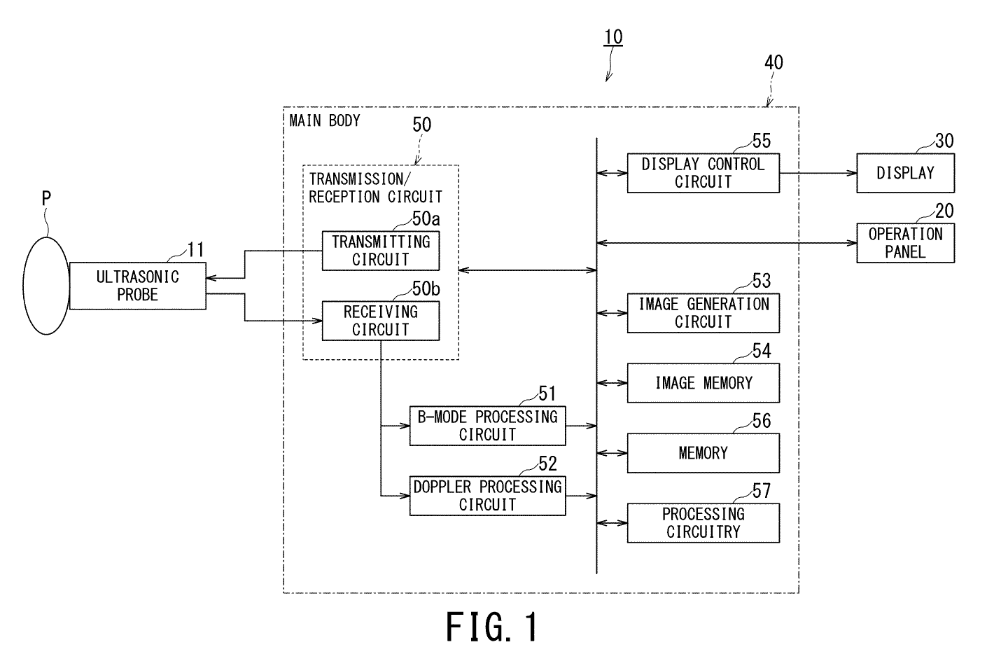

FIG. 1 is a block diagram showing an example of a configuration of the ultrasonic diagnostic apparatus 10 according to an embodiment of the present invention. The ultrasonic diagnostic apparatus 10 includes an ultrasonic probe 11, an operation panel 20, a display 30, and a main body 40.

The ultrasonic probe 11 is equipped with plural ultrasonic transducers (piezoelectric vibrators). Each of those plural ultrasonic transducers generates an ultrasonic wave on the basis of a drive signal supplied from the main body 40. The ultrasonic probe 11 transmits ultrasonic waves generated by the ultrasonic transducers to inside of a body of an object P, and receives echo signals from the object P so as to convert the echo signals into electric signals. Moreover, the ultrasonic probe 11 includes components such as a matching layer provided on the ultrasonic transducers and a backing material which prevents ultrasonic waves from propagating toward the back side of the ultrasonic transducers.

When the ultrasonic beam is transmitted from the ultrasonic probe 11 to the object P, the transmitted ultrasonic beam is successively reflected on the discontinuous surface of the acoustic impedance in the internal tissue of the object P, and the reflected waves are received as echo signals by the plural ultrasonic transducers. The amplitude of the received echo signal depends on the difference in the acoustic impedance at the discontinuous surface where the ultrasonic beam is reflected. When the transmitted ultrasonic pulse is reflected on the surface such as the moving blood flow or the heart wall, the reflected wave signal is subject to frequency deviation due to the Doppler effect, depending on the velocity component of the moving object with respect to the ultrasonic transmission direction.

The operation panel 20 functions as a touch command screen, and includes a display, a touch input circuit disposed beside this display, and a hardware key. The touch input circuit provides the main body 40 with information on an instruction position on a touch input circuit touched by a user. A keyboard, a mouse, a foot switch, a track ball, various types of buttons and the like can be used as the hardware key. The touch input circuit and the hardware key integrally constitute an input circuit which receives various types of commands from a user of the ultrasonic diagnostic apparatus 10.

The display 30 is configured of a general display output device such as a liquid crystal display and an OLED (Organic Light Emitting Diode) display, and display an ultrasonic image generated by the main body 40. Additionally, the display 30 displays an image for a user of the ultrasonic diagnostic apparatus 10 to input various types of commands with the use of the operation panel 20. Further, the display 30 displays notification information for a user received from the main body 40.

The main body 40 generates an ultrasonic image on the basis of an echo signal from the object P received by the ultrasonic probe 11. As shown in FIG. 1, the main body 40 includes a transmission/reception circuit 50, a B-mode processing circuit 51, a Doppler processing circuit 52, an image generation circuit 53, an image memory 54, a display control circuit 55, memory 56, and processing circuitry 57.

The transmission/reception circuit 50 includes a transmitting circuit 50a and a receiving circuit 50b. The transmission/reception circuit 50 controls transmission directivity and reception directivity in the transmission and reception of the ultrasonic waves, causes the ultrasonic probe 11 to transmit the ultrasonic wave to the object P, and generate the echo data on the basis of the echo signal received by the ultrasonic probe 11.

Further, the frame rate or the volume rate of the ultrasonic scanning set by the transmission/reception circuit 50 is controlled by the processing circuitry 57.

The ultrasonic diagnostic apparatus 10 according to the present embodiment can be applied to a case where the object P is scanned three dimensionally by the ultrasonic probe 11 as a two-dimensional ultrasonic probe in which a plurality of piezoelectric transducers are arranged two-dimensionally in a lattice pattern, to a case where the object P is scanned two dimensionally by the ultrasonic probe 11 as a one-dimensional ultrasonic probe in which a plurality of piezoelectric transducers are arranged in a row, and also can be applied to a case where the object P is scanned three dimensionally by rotating the one-dimensional ultrasonic probe.

In the following description, the ultrasonic probe 11 is the one-dimensional ultrasonic probe in which the plurality of piezoelectric transducers are arranged in a row, and an example in a case where the frame rate of the ultrasonic scanning is controlled by the processing circuitry 57 will be described. When the ultrasonic probe 11 is configured to be capable of three-dimensional scanning, the processing circuitry 57 preferably controls the volume rate of the ultrasonic scanning.

The transmitting circuit 50a includes a pulse generator, a transmission delay circuit, and a pulsar circuit, and supplies the ultrasonic probe 11 with a driving signal. The pulse generator repeatedly generates a rate pulse for forming an ultrasonic wave to be transmitted at a predetermined rate frequency. The transmission delay circuit focuses the ultrasonic wave generated from the ultrasonic probe 11 into a beam and provides, to each rate pulse generated by the pulse generator, a delay time per ultrasonic transducer that is necessary to determine the transmission directionality. Additionally, the pulsar circuit applies a driving pulse to the ultrasonic probe 11 at a timing based on each rate pulse. The transmission delay circuit changes the delay time provided to each rate pulse so as to appropriately adjust a transmission direction of the ultrasonic beam transmitted from the surface of the ultrasonic transducers.

Further, in order to execute a predetermined scan sequence under the control of the processing circuitry 57, the transmitting circuit 50a has a function of instantaneously changing parameters such as a transmission frequency and a transmission driving voltage. The function of changing a transmission driving voltage is implemented by a linear amplifier type of oscillator capable of instantaneously changing the value of the transmission driving voltage or a structure of electrically switching plural power-supply units.

The receiving circuit 50b includes an amplifier circuit, an A/D converter, and an adder circuit. The receiving circuit 50b receives echo signals received by the ultrasonic probe 11 and generates the echo data by performing various types of processing on the echo signals. The amplifier circuit performs gain correction processing by amplifying the echo signals for each channel. The A/D converter performs A/D conversion on the echo signals subjected to the gain correction processing, and provides the digitized data with a delay time necessary for determining reception directivity. The adder circuit performs addition processing of the echo signals digitized by the A/D converter so as to generate the echo data. Each reflected component from a direction according to reception directivity of each echo signal is enhanced by the addition processing of the adder circuit.

In the present embodiment, the transmitting circuit 50a can transmit a two-dimensional ultrasonic beam to the object P from the ultrasonic probe 11. Further, the receiving circuit 50b can generate two-dimensional echo data from the two-dimensional echo signal received by the ultrasonic probe 11. In addition, the processing circuitry 57 may generate volume data, on the basis of a plurality of two-dimensional echo data acquired at a predetermined frame rate while the ultrasonic probe 11 moves, and on the basis of the position information of the ultrasonic probe 11 at the time of acquiring each echo data.

The B-mode processing circuit 51 receives echo data from the receiving circuit 50b and performs logarithmic amplification, envelope detection on the echo data, and the like, so as to generate (B-mode) data expressing the signal intensity by luminance.

The Doppler processing circuit 52 performs frequency analysis on velocity information from the echo data received from the receiving circuit 50b, and extracts a blood-flow component, a tissue component, and a contrast-agent echo component by the Doppler effect. In this manner, the Doppler processing circuit 52 generates data (Doppler data) in which moving-object information items such as the average velocity, variance, and power are extracted for multiple points.

The image generation circuit 53 generates ultrasonic image data on the basis of the echo signals received by the ultrasonic probe 11. For example, the image generation circuit 53 generates two-dimensional B-mode image data in which intensity of each reflected wave is indicated by luminance on the basis of two-dimensional B-mode data generated by the B-mode processing circuit 51. Additionally, the image generation circuit 53 generates image data of a two-dimensional color Doppler image as an average velocity image, a variance image, a power image, a combination image of these images, or the image indicative of the moving-object information, on the basis of the two-dimensional Doppler data generated by the Doppler processing circuit 52.

The image memory 54 is a memory circuitry configured to store data of the two-dimensional ultrasonic images generated by the processing circuitry 57.

The display control circuit 55 includes a GPU (Graphics Processing Unit), a VRAM (Video RAM), and the like, and is controlled by the processing circuitry 57 to display an image requested for display output from the processing circuitry 57 on the display 30. The display control circuit 55 may display an image, which is equivalent to the image displayed on the display 30, on the display of the operation panel 20.

The memory 56 is equipped with a configuration including memory media which can be read by a processor such as a magnetic memory medium, an optical memory medium, and a semiconductor memory. The memory 56 may be configured such that some or all of the programs and data stored in those memory media can be downloaded by means of communication via an electronic network.

The memory 56 is controlled by the processing circuitry 57 and, each time the frame rate or the volume rate is changed, stores the image data in a different file. Further, when the processing circuitry 57 has the peak hold function, the memory 56 may store the B-mode data and the Doppler data before the peak hold processing is executed.

The processing circuitry 57 is a processor configured to controls the entire operation of the ultrasonic diagnostic apparatus 10, and execute, by reading out and executing the program stored in the memory 56, a procedure for changing the frame rate or the volume rate of the ultrasonic scanning according to the dynamics of the contrast agent.

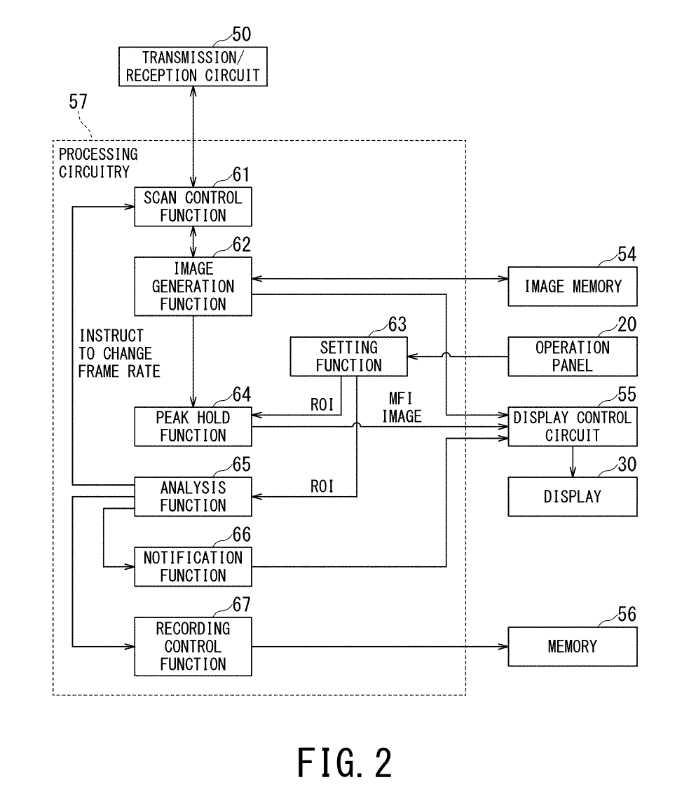

FIG. 2 is the schematic block diagram illustrating functions of processor of the processing circuitry 57. As shown in FIG. 2, the processor of the processing circuitry 57 implements a scan control function 61, an image generation function 62, a setting function 63, a peak hold function 64, an analysis function 65, a notification function 66, and recording control function 67. Each of these functions is stored in the memory 56 in the form of a program.

The scan control function 61 controls the transmission/reception circuit 50 to change the frame rate or the volume rate of the ultrasonic scanning according to the analysis result of the dynamics of the contrast agent by the analysis function 65.

The image generation function 62 generates an ultrasonic image to be displayed on the display 30 on the basis of the image data (hereinafter referred to as image data acquired by the ultrasonic scanning) generated by the image generating circuit 53 according to the echo data acquired by the ultrasonic scanning.

The setting function 63 sets the region of interest 72 in the ultrasound image on the basis of the user instruction via the operation panel 20.

The peak hold function 64 executes a processing (peak hold processing) of holding the highest value of the pixel value for each of the pixels belonging to the region of interest 72 on the basis of the image data repeatedly acquired by the ultrasonic scanning. It is preferable to store the B-mode data and the Doppler data before executing the peak hold processing in the memory 56. Then, the peak hold function 64 forms an image of the region of interest 72 using the highest value of this pixel value, and displays it on the display 30. When the flash is executed, the peak hold function 64 resets the highest value of the pixel value held for each pixel belonging to the region of interest 72. The ultrasonic diagnostic apparatus 10 does not have to include the peak hold function 64.

Note that the peak hold processing (also referred to as MFI, Micro Flow Imaging) is a processing of holding the luminance that reaches the highest luminance for each pixel. As a result, even if the number of bubbles in blood vessels varies for each frame, the blood vessels running are clarified by overlapping the frames since the luminance value of each pixel is the highest value up to the present time.

The analysis function 65 analyzes the dynamics of the contrast agent in the region of interest 72 of the object P on the basis of the image data acquired by the ultrasonic scanning. The scan control function 61 changes the frame rate or the volume rate of the ultrasonic scanning according to the analysis result of the dynamics of the contrast agent by controlling the transmission/reception circuit 50.

When the transmission/reception circuit 50 changes the frame rate or the volume rate, the notification function 66 causes the display 30 to display information to that effect, information on the current frame rate, and the like.

The recording control function 67 stores the image data acquired by the ultrasonic scanning in the memory 56 such that, each time the transmission/reception circuit 50 changes the frame rate or the volume rate, the image data is stored in a different file.

Next, an example of the operation of the ultrasonic diagnostic apparatus 10 and the controlling method according to the present embodiment will be described.

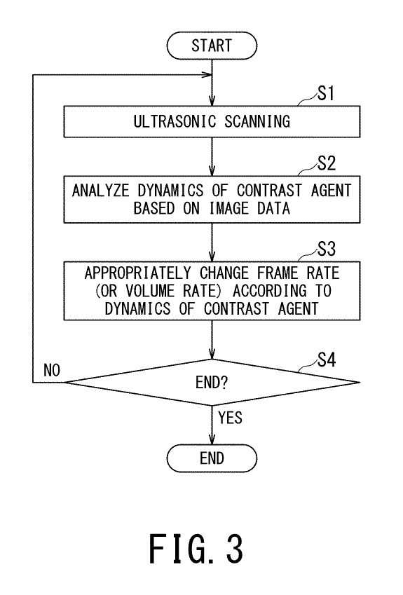

First, the outline of the operation of the ultrasonic diagnostic apparatus 10 and the controlling method according to the present embodiment will be described with reference to FIG. 3.

FIG. 3 is a flowchart showing an example of a schematic procedure for changing the frame rate or the volume rate of the ultrasonic scanning according to the dynamics of the contrast agent by the processing circuitry 57 shown in FIG. 1. A reference character with “S” followed by a number in FIG. 3 denotes each step of the flowchart.

First, in step S1, the transmission/reception circuit 50 performs the ultrasonic scanning on the object P to which the contrast agent is administered.

Next, in step S2, the analysis function 65 analyzes the dynamics of the contrast agent on the basis of the image data acquired by the ultrasonic scanning.

Next, in step S3, the scan control function 61 appropriately changes the frame rate or the volume rate according to the dynamics of the contrast agent analyzed and determined by the analysis function 65.

Next, in step S4, the processing circuitry 57 determines whether or not the ultrasonic scanning should be ended. For example, when an instruction by the user to end the ultrasonic scanning via the operation panel 20 is received, it is determined that the operation should be ended, and the series of procedures is terminated. Meanwhile, when it is determined that the process should not be terminated, the process returns to step S1.

According to the above procedure, instead of changing the frame rate every predetermined constant time, the frame rate or the volume rate of the ultrasonic scanning can be changed according to the dynamic of the contrast agent.

Subsequently, a method of changing the frame rate or the volume rate of the ultrasonic scanning according to the dynamics of the contrast agent will be described in detail. In the following description, an example will be described in the case where the frame rate of the ultrasonic scanning is controlled by the processing circuitry 57.

First, the operation immediately after the injection of the contrast agent will be described in detail.

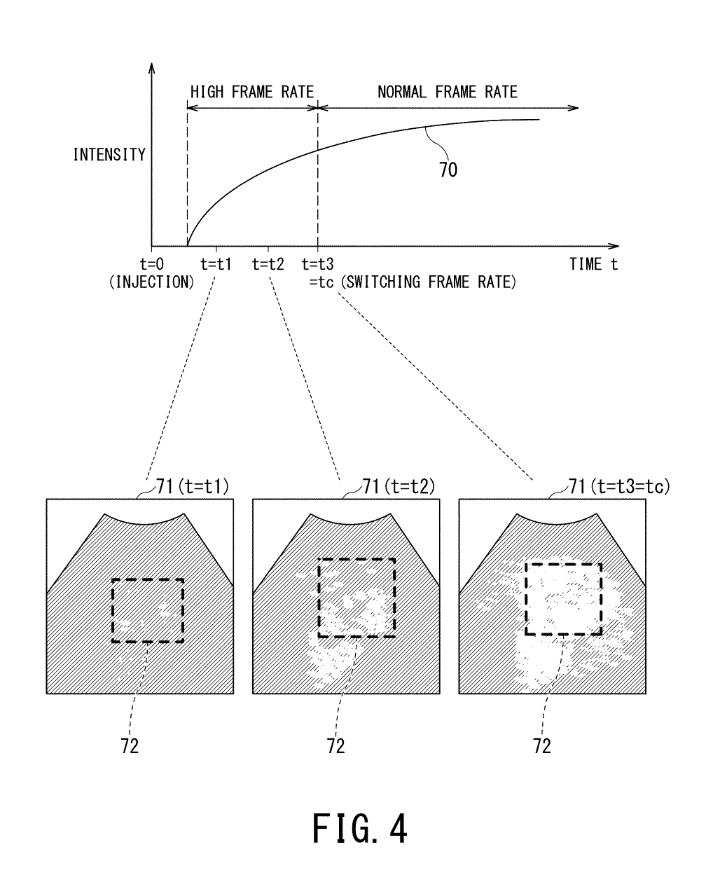

FIG. 4 is an explanatory diagram showing an example of a relationship between a time intensity curve (hereinafter referred to as TIC) 70 and the ultrasonic images 71, from immediately after the injection of the contrast agent until a predetermined time elapses.

As described above, the high temporal resolution imaging method with the high frame rate is desired in order to precisely observe the fast inflow process of the arterial predominant phase occurring immediately after the injection of the contrast agent. Meanwhile, after the inflow of the contrast agent is completed, a normal frame rate is suitable for preventing unnecessary destruction of the contrast agent and for observing the static post vascular phase. Hence, it is preferable to acquire data at the frame rate having the value according to the dynamics of the contrast agent, i.e., according to whether the contrast agent is in the fast inflow process of arterial predominant phase or in the completion state of the inflow such as post vascular phase.

As shown in FIG. 4, it is possible to analyze the dynamics of the contrast agent on the basis of the change in the image of the region of interest 72 of the ultrasound image 71. For example, it is considered that the region of interest 72 in the ultrasonic images 71 after the lapse of time t1 and after the lapse of time t2 from the injection of the contrast agent are in the inflow process of the contrast agent. On the other hand, the region of interest 72 after the lapse of time t3 from the injection of the contrast agent is considered that the main inflow of the contrast agent has been completed. Therefore, the time t=t3 is considered to be suitable as the timing tc for switching from the high frame rate (the first frame rate) to the normal frame rate (the second frame rate). The analysis function 65 determines the timing tc at which the frame rate is switched on the basis of the change in the image data of the region of interest 72 of the ultrasound image 71. Incidentally, the timing at which transition from the arterial predominant phase to the portal predominant phase occurs can be used as an example of the timing at which the main inflow of the contrast agent is completed.

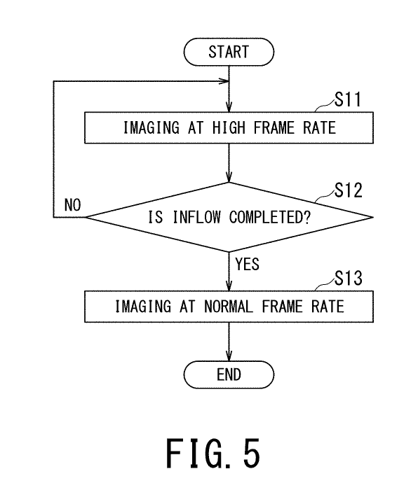

FIG. 5 is a flowchart showing an example of a procedure implemented by the processing circuitry 57 shown in FIG. 1 for changing the frame rate or the volume rate of the ultrasonic scanning according to the dynamics of the contrast agent immediately after the injection of the contrast agent.

The procedure shown in FIG. 5 is started immediately after the contrast agent is injected to the object P.

First, in step S11, the scan control function 61 controls the transmission/reception circuit 50 so as to perform the ultrasonic scanning at the high frame rate.

Next, in step S12, the analysis function 65 determines whether or not the inflow of the contrast agent into the region of interest 72 is completed on the basis of the image data of the region of interest 72 of the ultrasound image 71. When it is analyzed that the inflow of the contrast agent in the region of interest 72 is completed (YES in step S12), the scan control function 61 controls the transmission/reception circuit 50 so as to switch to the normal frame rate and to perform the ultrasonic scanning (step S13). When it is analyzed that the contrast agent is in the inflow process in the region of interest 72 (NO in step S12), the scan control function 61 controls the transmission/reception circuit 50 so as to perform the ultrasonic scanning as it is at the high frame rate.

According to the above procedure, the ultrasonic scanning at the high frame rate is performed immediately after administration of the contrast agent, and when it is analyzed that the inflow of the contrast agent in the region of interest 72 is completed on the basis of the dynamics of the contrast agent, the frame rate is automatically switched to the normal frame rate.

Three examples of a method of determining whether or not the flow of the contrast agent into the region of interest 72 is completed on the basis of the image data of the region of interest 72 of the ultrasound image 71 will be described.

The first determination method is a method of determining whether or not the inflow of the contrast agent into the region of interest 72 is completed on the basis of the luminance histogram 73 of the region of interest 72.

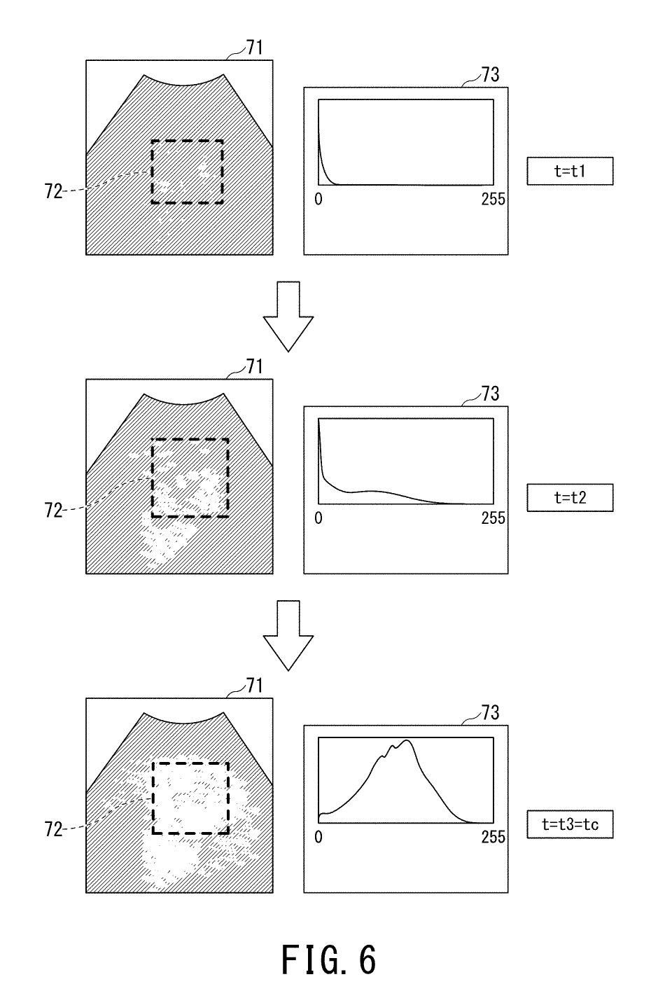

FIG. 6 is an explanatory diagram showing an example of a relationship between the ultrasonic image 71 at each of t=t1, t2, and t3 shown in FIG. 4 and the luminance histogram 73 of the region of interest 72. As shown in FIG. 6, the luminance histogram 73 of the region of interest 72 changes in accordance with the dynamics of the contrast agent. Therefore, the analysis function 65 generates the histogram on the pixel values in the region of interest 72, i.e., the luminance histogram 73, on the basis of the image data acquired by the ultrasonic scanning. And when the center of gravity of the luminance histogram 73 exceeds the threshold value, the analysis function 65 determines that the inflow of the contrast agent has been completed in the region of interest 72.

Further, whether the inflow of the contrast agent into the region of interest 72 is completed or not may be determined depending on the shape of the luminance histogram 73, for example, depending on whether the luminance histogram 73 has the local maximum value or not. As the method of determining whether the inflow of the contrast agent into the region of interest 72 is completed according to the shape of the luminance histogram 73, in addition to using the presence or absence of the maximum value, in addition to using the presence or absence of the local maximum value, convolutional neural network (hereinafter referred to as CNN) may be used, which is one of the deep learning used in recent years in the field of image recognition. For using CNN, the neural network learns the shape of the histogram as shown in the bottom row of FIG. 6 in advance.

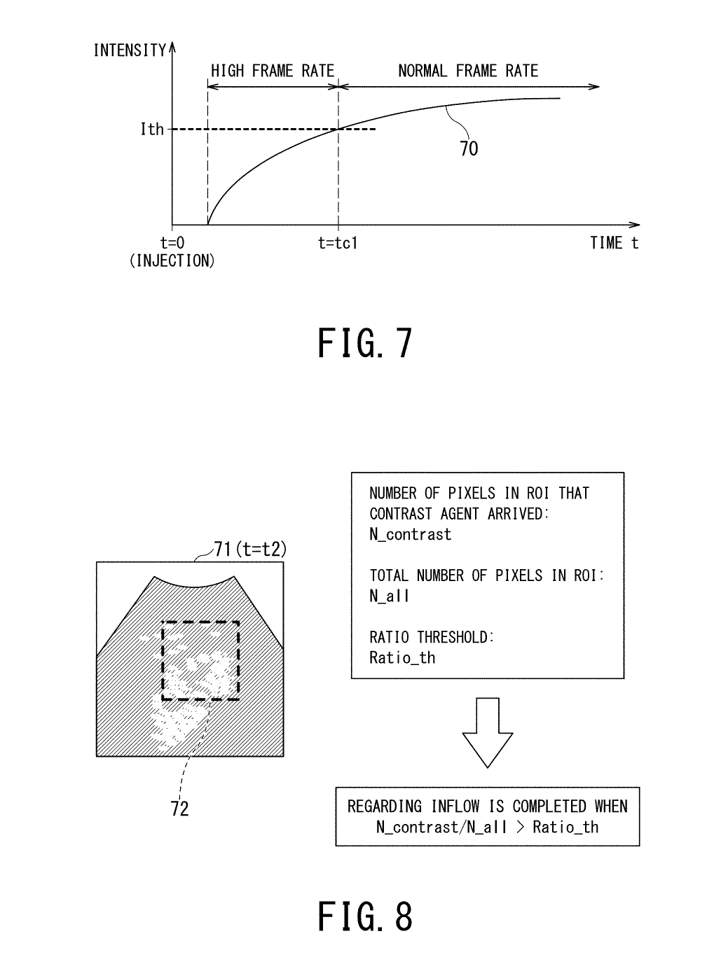

The second determination method is a method of determining whether or not inflow of the contrast agent into the region of interest 72 is completed on the basis of the TIC 70 of the predetermined pixel in the region of interest 72. FIG. 7 is an explanatory diagram showing an example of a relationship between TIC 70 and the threshold value Ith of the signal intensity. As shown in FIG. 7, the timing tc1 may be determined on the basis of the signal intensity of the TIC 70. In this case, the analysis function 65 calculates the representative value of the pixel value in the region of interest 72 on the basis of the image data acquired by the ultrasonic scanning. The analysis function 65 determines that the timing tc1 to switch the frame rate has come when the signal intensity of the time curve (TIC 70) of the representative value exceeds the threshold Ith. The threshold value Ith may be set to, for example, 10 dB from the base line.

In hepatocellular carcinoma (HCC), TIC 70 peaks after a predetermined time has elapsed since the inflow of the contrast agent started, and TIC 70 does not recover from washout state despite waiting more than 10 minutes and the time belonging to the post vascular phase, because there is no Kupffer cell. That is, when the TIC 70 has a peak, it may be determined that the time when the peak has come is the timing tc to switch the frame rate.

In addition, even when the ultrasonic diagnostic apparatus 10 has the peak hold function 64, the second determination method can be applied when the B-mode data or the Doppler data before the peak hold processing is stored in the memory 56.

The third determination method is a method of determining whether or not the inflow of the contrast agent into the region of interest 72 is completed on the basis of the number of pixels in the region of interest 72 to which the contrast agent has reached.

FIG. 8 is a diagram for explaining the third inflow completion determination method. A pixel having a luminance value equal to or larger than a predetermined luminance value is defined as a pixel reached by the contrast agent. It can be considered that the inflow of the contrast agent into the region of interest 72 has been completed when almost the entire area of the region of interest 72 is occupied by the pixels having the luminance value equal to or larger than the predetermined luminance value. Thus, assuming N_contrast is the number of pixels counted as the pixels reached by the contrast agent in the region of interest 72, N_all is the total number of pixels in the region of interest 72, and Ratio_th is the ratio threshold, then it may be considered that the inflow of the contrast agent into the region of interest 72 is completed when N_contrast/N_all>Ratio_th is satisfied.

Further, when the total number of pixels N_all of the region of interest 72 is given in advance, the threshold value N_th of the number of pixels is set corresponding to N_all, and it may be considered that the inflow of the contrast agent into the region of interest 72 is completed when N_contrast>N_th is satisfied.

According to any one of these three methods, it is possible to determine whether or not the inflow of the contrast agent has been completed in the region of interest 72 on the basis of the image data of the region of interest 72 of the ultrasound image 71, and to determine the timing tc to switch (change) the frame rate.

Next, a method of changing the frame rate in the case of executing the flash for sweeping out the bubbles of the contrast agent in the scanning area will be described.

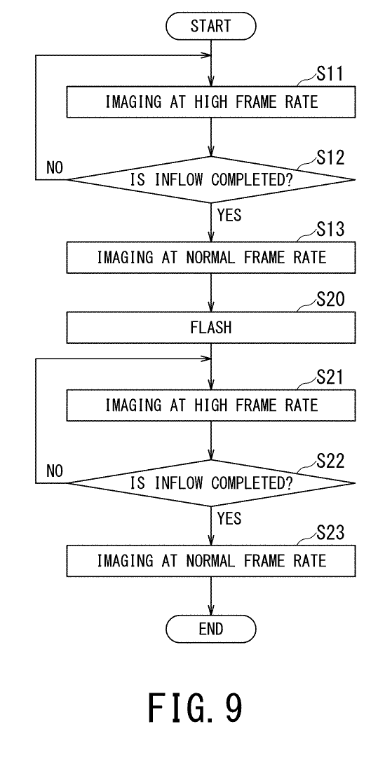

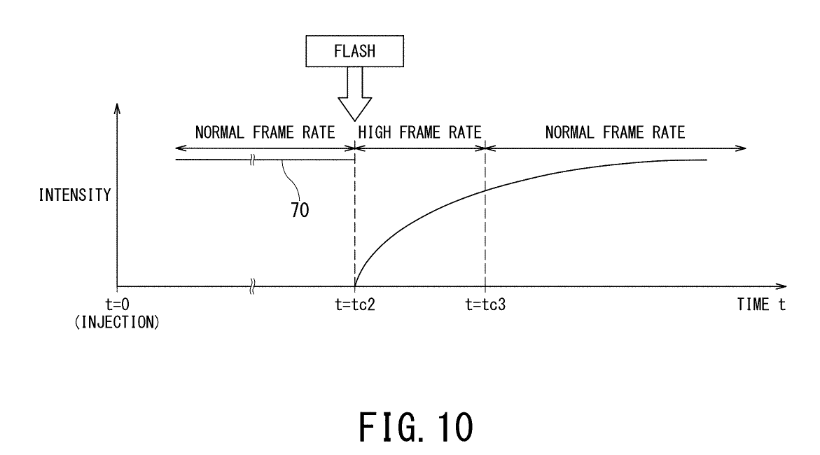

FIG. 9 is a flowchart showing an example of a procedure by processing circuitry 57 shown in FIG. 1 for changing the frame rate or the volume rate of the ultrasonic scanning according to the dynamics of the contrast agent before and after the execution of the flash. Steps equivalent to those in FIG. 5 are denoted by the same reference numerals and the explanations of which are omitted. FIG. 10 is an explanatory diagram showing an example of TIC before and after the execution of the flash.

This procedure starts with the transition to the post vascular phase after the predetermined time has elapsed from the injection of the contrast agent.

First, in accordance with the procedure shown in FIG. 5, in step 13, the scan control function 61 controls the transmission/reception circuit 50 so as to switch to the normal frame rate at t=tc and perform the ultrasonic scanning.

Next, in step S20 the flash is executed. Here, the flash is executed to sweep out the bubbles of the contrast agent in the scanning area with high sound pressure. When the observation site is the liver, for example, the ultrasonic diagnostic apparatus 10 according to the embodiment can be observe, with low sound pressure at normal frame rate, the bubbles in the peripheral blood vessels perfusing throughout the liver, when the predetermined time has elapsed after the injection of the contrast agent and the portal predominant phase has come. However, the tumor vessels of the lesion of interest are buried, and it becomes difficult to observe. To cope with this, by executing the flash to sweep out the bubbles in the scanning area at high sound pressure on or after the portal predominant phase, for example in the post vascular phase, it can be observed how the reflux flow occurs. Note that the contrast agent may be re-injected at the same time as or before the flash.

For example, the signal intensity of the TIC 70 returns to substantially zero when the flash is executed according to the instruction via the operation panel 20 by the user (see FIG. 10). Thus, the scan control function 61 controls the transmission/reception circuit 50 so as to switch to the high frame rate again at the timing t=tc2 of the flash and perform the ultrasonic scanning.

When the ultrasonic diagnostic apparatus 10 has the peak hold function 64, the peak hold function 64 resets the highest value of the pixel value held for each pixel belonging to the region of interest 72 when the flash is executed.

Next, in step S22, as in step S12 of FIG. 5, the analysis function 65 determines whether or not the inflow of the contrast agent into the region of interest 72 is completed on the basis of the image data of the region of interest 72 of the ultrasound image 71. When it is analyzed that the inflow of the contrast agent in the region of interest 72 has been completed (YES in step S22, see t=tc3 in FIG. 10), the scan control function 61 controls the transmission/reception circuit 50 so as to switch to the normal frame rate and perform the ultrasonic scanning (step S23). Meanwhile, when it is analyzed that the contrast agent is in the inflow process in the region of interest 72 (NO in step S22), the scan control function 61 controls the transmission/reception circuit 50 so as to perform the ultrasonic scanning as it is at the high frame rate. Any of the above three methods may be used as a method of determining whether or not the flow of the contrast agent into the region of interest 72 is completed on the basis of the image data of the region of interest 72.

According to the above procedure, it is possible to change the frame rate or the volume rate of the ultrasonic scanning according to the dynamics of the contrast agent, and it is also possible to surely change the ultrasonic scanning to the high frame rate with the execution of flash.

When the transmission/reception circuit 50 changes the frame rate or the volume rate, the notification function 66 may display the information indicating the change, the information on the current frame rate, and the like on the display 30. The user can surely recognize that the frame rate has been changed by confirming these displays.

Further, each time the transmission/reception circuit 50 changes the frame rate or the volume rate, the recording control function 67 may store the image data in the different file. It makes post processing extremely easy with storing the image data in a separate file every time the frame rate is changed. The timing to start storing regarding immediately after the injection of the contrast agent may be the timing at which an instruction via the operation panel 20 by the user is accepted or may be the timing at which it is analyzed that the inflows of the contrast agent into the region of interest 72 has started.

Also, it is sufficient to distinguish the image data belonging to the same scanning period that is delimited by the switching timing of the frame rate, and the image data of different period may not be necessarily stored in a separate file. More specifically, for example, image data of all the periods may be stored in the same file and supplementary information capable of identifying the timing of the change of the frame rate, e.g., a bookmark attached to moving image data, may be added to the image data.

According to at least one of the above-described embodiments, the frame rate or the volume rate of the ultrasonic scanning can be changed according to the dynamics of the contrast agent.

The processing circuitry 57 in the above-described embodiments is an example of the processing circuitry described in the claims.

In addition, the term “processor” used in the explanation in the above-described embodiments, for instance, refer to circuitry such as dedicated or general purpose CPUs (Central Processing Units), dedicated or general-purpose GPUs (Graphics Processing Units), or ASICs (Application Specific Integrated Circuits), programmable logic devices including SPLDs (Simple Programmable Logic Devices), CPLDs (Complex Programmable Logic Devices), and FPGAs (Field Programmable Gate Arrays), and the like. The processor implements various types of functions by reading out and executing programs stored in the memory circuitry.

Although in the above-described embodiments an example is shown in which the processing circuitry configured of a single processor implements every function, the processing circuitry may be configured by combining plural processors which are independent from each other and each processor implements each function of the processing circuitry by executing corresponding program. When a plurality of processors are provided for the processing circuitry, the memory medium for storing programs may be individually provided for each processor, or one memory circuitry may collectively store programs corresponding to all the functions of the processors.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising:
a transmission/reception circuit configured to repeatedly perform ultrasonic scanning of an object to which a contrast agent is administered; and
processing circuitry configured to analyze dynamics of the contrast agent in a region of interest in the object based on image data acquired by the ultrasonic scanning,
wherein the transmission/reception circuit performs the ultrasonic scanning at a frame rate or a volume rate having a value according to an analysis result of the processing circuitry.

2. The apparatus according to claim 1, wherein the processing circuitry is configured to:
generate a histogram on pixel values in the region of interest based on the image data; and
analyze the dynamics of the contrast agent based on the histogram.

3. The apparatus according to claim 1, wherein the processing circuitry is configured to:
calculate a representative value of pixel values in the region of interest based on the image data; and
analyze the dynamics of the contrast agent based on the representative value.

4. The apparatus according to claim 1, wherein the processing circuitry configured to:
set the region of interest in an image generated based on the image data;
count a number of pixels in the region of interest having pixel values equal to or larger than a predetermined value based on the image data; and
analyze the dynamics of the contrast agent based on a result of the counting.

5. The apparatus according to claim 1, wherein:
the processing circuitry is configured to analyze, based on the image data, at least whether the region of interest is in a state where the contrast agent is in the inflow process or the inflow of the contrast agent is completed; and
the transmission/reception circuit is configured to perform the ultrasonic scanning at a first frame rate or a first volume rate when the contrast agent is analyzed as being in the inflow process in the region of interest, and perform the ultrasonic scanning at a second frame rate or a second volume rate lower than the first frame rate or the first volume rate when the inflow of the contrast agent in the region of interest is analyzed as being completed.

6. The apparatus according to claim 5, wherein, when executing a flash for sweeping out bubbles of the contrast agent in a scanning area while performing the ultrasonic scanning at the second frame rate or the second volume rate, the transmission/reception circuit is configured to perform the ultrasonic scanning at the first frame rate or the first volume rate by switching from the second frame rate or the second volume rate as the flash is executed.

7. The apparatus according to claim 6, wherein, after switching to the first frame rate or the first volume rate with the execution of the flash, when the inflow of the contrast agent in the region of interest is analyzed as being completed by the processing circuitry, the transmission/reception circuit is configured to perform the ultrasonic scanning at the second frame rate or the second volume rate by switching from the first frame rate or the first volume rate.

8. The apparatus according to claim 1, wherein the processing circuitry is configured to:
hold a highest value of a pixel value for each pixel in the region of interest based on the image data repeatedly acquired by the ultrasonic scanning; and
generate an image of the region of interest using the highest value of the pixel value and display the generated image on a display;

9. The apparatus according to claim 6, wherein the processing circuitry is configured to:
hold a highest value of pixel values for each pixel in the region of interest based on the image data repeatedly acquired by the ultrasonic scanning;
generate an image of the region of interest using the highest value of the pixel value and display the generated image on a display; and
when the flash is executed, reset the highest value of the pixel value held for each pixel in the region of interest.

10. The apparatus according to claim 1, wherein, when the transmission/reception circuit changes the frame rate or the volume rate, the processing circuitry is configured to display information to that effect on a display.

11. The apparatus according to claim 1, wherein the processing circuitry is configured to make a memory store the image data acquired by the ultrasonic scanning, and each time the transmission/reception circuit changes the frame rate or the volume rate, make the memory store the image data in a different file.

12. The apparatus according to claim 5, wherein the processing circuitry is configured to determine that the contrast agent is in the inflow process during an arterial predominant phase, and determine that the inflow of the contrast agent is completed when transitioning from an arterial predominant phase to a portal predominant phase.

13. A controlling method, comprising:
repeatedly performing ultrasonic scanning of an object to which a contrast agent is administered;
analyzing dynamics of the contrast agent in a region of interest in the object based on image data acquired by the ultrasonic scanning; and
changing a value of a frame rate or a volume rate of the ultrasonic scanning according to an analysis result of dynamics of the contrast agent.
